(19) United States
(12) Patent Application Publication
(10) Pub. No.: US 2008/0306132 A1
(43) Pub. Date: Dec. 11, 2008

(54) Novel oral compositions of carporen

(76) Inventors: Praveen Hiremath, Cockeysville, MD (US); Gerry Macedo, Bradbury, CA (US)

Correspondence Address: Jay R Akhave, # 612, 2058 N Mills Ave, Claremont, CA, 91711, US

(21) Appl. No.: 12/157,385

(22) Filed: Jun. 9, 2008

Related U.S. Application Data
(60) Provisional application No. 60/933,504, filed on Jun. 7, 2007.

(51) Int. Cl. A61K 31/403

(52) U.S. Cl. 514/411

(57) ABSTRACT

An improved semisolid composition for an oral veterinary application and a method of making it. A carprofen paste composition is made for orally administering to animals using calibrated syringe dispensers for exact dosages.

### FIELD OF INVENTION

The present invention relates to a new simpler way of making an oral paste with carprofen as an active pharmaceutical ingredient in a veterinary application. This oral paste is specifically designed to be orally administered using a calibrated syringe.

The process provides for a simple thickening of carprofen solution with polymeric thickeners. The thickened paste exhibits good stability and consistency. The mouth appeal of such pastes enables improved animal compliance as opposed to solid viscosity modifiers such as fumed silica.

### BACKGROUND OF THE INVENTION

It is well known to artisans of ordinary skill in this field, e.g., veterinarians, that the canine species, i.e., dogs, especially older dogs, are very susceptible to chronic inflammatory processes such as degenerative joint disease. The pharmaceutical agents which have been proven beneficial in the treatment of such conditions are non-steroidal anti-inflammatory drugs (NSAIDs), and one such useful NSAID is carprofen. This drug is a member of the class of drugs that includes indomethacin, naproxen and ketoprofen. Chemically, carprofen is 6-chloro-.alpha.-methyl-9H-carbazole-2-acetic acid.

Carprofen has been found to be very effective therapeutically when administered orally and parenterally. U.S. Pat. No. 6,506,785 to Pfizer teaches the use of carporfen and its derivates for treating and preventing the early stages of degeneration of articular cartilage or subchondral bone in mammals. Hence horses, dogs cats can be treated with carprofen and its derivatives to obtain such therapeutic results.

Currently, both oral (tablets) formulations are available in the US Market. For example, oral dosage forms for the pharmaceutical active ingredient Rimadyl (generic carprofen) that are limited to tablets and chewable tablets. These tablets can contain flavoring agents. Carprofen is often administered orally to animals, such as dogs with such tablets.

Oral route of drug delivery enjoys several advantages in terms of patient compliance, patient comfort and cost over other routes, a fact also true in case of animals as well as humans. However, our search to date has not revealed any semisolid composition in the veterinary market for carprofen oral administration that is readily available in the market.

Tablet dosage forms for carprofen are known. U.S. Pat. No. 6,497,899 to Pfizer teaches non-friable, rapidly disintegrating, fast-dissolving solid dosage forms that are produced from pharmaceutically acceptable steam extruded polymers and by example shows that pharmaceutically acceptable levels of carprofen can be loaded onto such a carrier base.

This dosage form can dissolve in the mouth for rapid drug delivery. This patent further teaches that the dosage forms of the invention dissolve in the mouth and are particularly useful for subjects that require or desire oral medication but have difficulty swallowing standard oral dosage forms or in subjects suffering from emesis or who require or desire a faster acting treatment, or a more palatable dosage form.

Solid dosage formulations, like tablets, although preferred and most widely used dosage forms in humans, they suffer from certain drawbacks particularly in animals. Solid dosage forms are not well tolerated and generally difficult to administer in animals.

US patent application 20030212123 discloses a method of treatment of pain and inflammation in dogs with anti-inflammatory agents which are non-steroidal anti-inflammatory drugs (NSAIDs), and in particular such agents having a reduced incidence of adverse gastrointestinal side effects, since such side effects are a prevalent and potentially severe problem in dogs. This patent :application further discloses the compositions for treating such diseases.

US patent application 20030007958 teaches a pharmaceutical or veterinary paste formulation comprising: an effective amount of a therapeutic agent; fumed silica; a viscosity modifier; a hydrophilic carrier; optionally, an absorbent; and optionally, a colorant, stabilizer, surfactant, or preservative. The use of fumed silica however creates a negative mouth appeal affecting patient compliance. It would be very desirable to obtain a carprofen paste formulation that does not use fumed silica from a patient compliance viewpoint and yet have a stable formulation.

US patent application 20070042006 discloses a stable solvent-based composition of carprofen which is particularly useful in warm blooded animals such as dogs. The composition comprises a therapeutically effective amount of carprofen, one or more polyols, one or more stabilizing agents and optionally, one or more co-solvents. This patent application specifically discloses the stable solution (liquid) composition of carprofen. Further, this application teaches that to obtain the invented stable solution composition the stabilizer addition is essential which otherwise would lead to unstable formulations with unacceptable level of degradation. Also, the compositions described and disclosed are non-aqueous formulations. This patent highlites the need of stabilizing agents in ensuring stable carprofen formulations that do not degrade.

U.S. Pat. No. 6,506,785 also teaches the oral delivery of carprofen as a paste on the back of the tongue. Typical dosage forms and amounts would include besides others, include an oral administration of carprofen at a dose rate of 4.0 mg/kg/day of bodyweight as an oral paste syringed on the back of the tongue, given one hour before feeding. The patent does not however describe how to make such a stable paste in any particularity.

Although these applications mention that carprofen can also be formulated into a paste composition, none of the examples or patent applications as a whole disclose any aspect of the carprofen semisolid composition, its formulation and preparation aspects with any particularity. Rather, the main invention of 20030212123 application was to achieve selective inhibition of COX-2 enzyme which ultimately lead to better anti-inflammatory or analgesic efficacy and lower incidence of side effects. Further, 20030212123 application discloses chewable or implantable compositions as preferred formulations for controlled oral drug delivery. The patent application 20030007958 as a whole teaches the new crystalline polymorphic form (polymorph B) of firocoxib (also granted as U.S. Pat. No. 6,78,7342 with claims restricted to firocoxib only). Patent application 20070042006 specifically discloses liquid (solution) compositions of carprofen with stabilizers as an essential element in the formulation. Also, 20070042006 teaches that the carprofen is present in the composition in a solubilized form. Therefore, none of the above patents/applications describe, exemplify or claim oral semisolid compositions of carprofen. These applications do not motivate a person having ordinary skill in the art to arrive at the oral semisolid compositions of carprofen which will be discussed in the present invention.

Also, we could not come across any marketed oral semisolid composition of carprofen as revealed by our searches in FDA (The Green Book and VMRCVM) data base, Compendium of Veterinary Products (8^{th} edition), Veterinary Drug Handbook (4^{th} edition) and other available online data bases. Non-availability of any marketed semisolid carprofen composition further proves that it is not obvious to obtain a good quality and stable semisolid composition of carprofen without undue experimentation.

Hence, there is a market need for the development of an oral semisolid compositions of carprofen to aid in the convenient and easy administration of carprofen to animals without compromising its therapeutic activity. With a stable semisolid composition, a relatively precise dose of the drug can be administered in a pre-loaded calibrated syringe. This stability feature is extremely useful in the accurate and convenient dosage titration for administration into animals according to their body weights. The present inventors have developed a stable oral semisolid composition of carprofen. These compositions do not require the addition of special stabilizing agents for achieving stable compositions and also are devoid of fumed silica or any other additional ingredients as mentioned in prior art compositions. Also, carprofen is present in the composition as a fine homogeneously dispersed form as opposed to solubilized or dissolved form disclosed in prior art compositions. Further, the compositions of the present invention are cost effective, simple and could be easily developed on a commercial scale with minimum to moderate industrial manufacturing facilities and without complex manufacturing steps/operations.

### OBJECTIVE OF INVENTION

The main objective of the present invention is to provide a pharmaceutically effective carporfen paste to the market that has a minimal number of components for considerations of cost. It is yet an objective of the invention to formulate as stable formulation with such minimal components. It is another objective of this invention to provide a simple manufacturing method to make such a paste. It is an objective of this invention to have paste formulation that is devoid of silica which is a detractor for animal patient compliance.

### SUMMARY OF INVENTION

The inventors of the present invention obtained a novel oral semisolid composition of carprofen with particular combination of solvents and ingredients which is suitable for administration in animals.

According to one embodiment, there is a provided an oral semisolid composition comprising: a therapeutically effective amount of carprofen; water; one or more cosolvents; one or more hydrophilic gelling polymers; and optionally pharmaceutically acceptable excipients.

According to another embodiment, there is provided a method of treating pain and/or inflammation in an animal, the method comprising administering to the animal a oral semisolid composition comprising: a therapeutically effective amount of carprofen; water; one or more cosolvents; one or more hydrophilic gelling polymers; and optionally pharmaceutically acceptable excipients.

### DETAILED DESCRIPTION OF THE INVENTION

The invention relates to novel oral semisolid compositions of carprofen for administration in animals. These semisolid compositions can be improved alternatives to solid dosage forms and liquid formulations for oral delivery of carprofen in animals. The difficulty in administration associated with solid dosage forms and stability problems of liquid formulations can be minimized with the use of these novel semisolid compositions.

According to one embodiment, the invention describes an oral semisolid composition comprising: a therapeutically effective amount of carprofen; water; one or more cosolvents; one or more hydrophilic gelling polymers; and optionally pharmaceutically acceptable excipients.

Preferably, carprofen is included in the composition in an amount of about 0.1 to 10% w/w of the composition. More preferably, carprofen is present in an amount from about 1 to 5% w/w of the composition.

Water may be used in an amount of about 5 to 80% w/w of the composition. Preferably water is present from about 20 to 70% w/w of the composition.

The term “semisolid composition” as used herein refers to a cream, paste, gel, ointment or jelly.

One or more cosolvents used in the composition are those which contain hydroxyl groups or other polar groups and these cosolvents may be selected from one or more of propylene glycol, polyethylene glycol, polypropylene glycol, glycol ether, glycerol, polyoxyethylene alcohols and esters of polyoxyethylene fatty acids. Preferably, cosolvent is selected from one or more of propylene glycol and polyethylene glycol. Cosolvents may be used in an amount of about 5 to 80% w/w of the composition. Preferably, cosolvent is used in an amount of about 20 to 70% w/w of the composition.

One or more hydrophilic gelling polymers may be selected from one or more of carboxyvinyl polymers (carbomer) such as carbopol; acrylic copolymers such as acrylate/alkyl acrylate copolymers; polyacrylamides or polysaccharides such as xanthan gum, acacia, tragacanth gum, guar gum, karaya gum, alginates and gelatin; and modified celluloses such as carboxymethyl cellulose sodium, hydroxypropyl methylcellulose, hydroxyethyl cellulose and hydroxypropyl cellulose. Preferably, hydrophilic gelling polymer is selected from one or more of carbopol, hydroxyethyl cellulose, hydroxypropyl methylcellulose, carboxymethyl cellulose sodium and sodium alginate. Hydrophilic gelling polymer may be present in an amount of about 0.1 to 20% w/w of the composition. Preferably, hydrophilic gelling polymer is present in an amount of about 0.2 to 10% w/w of the composition.

The optional pharmaceutically acceptable excipients are those which aid in the development of proper and desired quality semisolid compositions. The optional pharmaceutically acceptable excipients may be selected from one or more of neutralizing agents such as triethanol amine, sodium hydroxide, ammonium hydroxide and potassium hydroxide; flavoring agents; sweeteners; coloring agent; opacifying agents such as titanium dioxide; and buffering agents.

The compositions of the present invention would further be considered for the routine product developmental activities according to official and standard regulatory guidelines with ultimate goal of reaching the market place with standard quality, safe and efficacious product. In brief, the compositions of present invention would be evaluated for their in-vitro release characteristics, long-term stability and other important qualities which are essential and vital requirements in the product development activities. Further, the optimized compositions would be considered for bioequivalence studies according standard protocol.

The invention can further be described by the following non-limiting examples.

### EXAMPLE 1

| S. No. | Ingredients | Quantity (% w/w) |
|---|---|---|
| 01 | Carbopol 974P | 0.3 |
| 02 | Propylene glycol | 36.0 |
| 03 | Triethanol amine | 0.2 |
| 04 | Carprofen | 2.0 |
| 05 | Purified water | Q.S. to 100% (61.3) |

### EXAMPLE 2

| S. No. | Ingredients | Quantity (% w/w) |
|---|---|---|
| 01 | Carbopol 974P | 0.5 |
| 02 | Propylene glycol | 47.5 |
| 03 | Triethanol amine | 0.3 |
| 04 | Carprofen | 2.0 |
| 05 | Purified water | Q.S. to 100% (49.5) |

Procedure for examples 1 and 2:
1) Carprofen was dissolved in propylene glycol by continuous stirring.
2) Carbopol was added to the drug solution of step 1 and mixed continuously to homogenously disperse it.
3) Purified water was added to step 2 with continuous stirring until a homogenous semisolid paste obtained.
4) Finally, triethanol amine was added to step 3 with continuous mixing and the composition was set aside for about 4 hours to get the final semisolid paste composition of desired consistency.

### EXAMPLE 3

| S. No. | Ingredients | Quantity (% w/w) |
|---|---|---|
| 01 | Hydroxypropyl | 4.0 |
| | methylcellulose | |
| 02 | Propylene glycol | 40.0 |
| 03 | Carprofen | 2.0 |
| 04 | Purified water | Q.S. to 100% (52.0) |

Procedure for example 3:
1) Carprofen was dissolved in propylene glycol by continuous stirring.
2) HPMC was added to the drug solution of step 1 and mixed continuously to homogenously disperse it.
3) Purified water was added to step 2 with continuous stirring until a homogenous semisolid gel was obtained.
4) The gel formed in step 3 was set aside for about 24 hours to get the final composition with required consistency.

We claim:

1. An oral semisolid composition for administering NSAIDs comprising:
a) a therapeutically effective active ingredient,
b) a cosolvent,
c) a hydrophyllic gelling polymer,
d) water.

2. A product according to claims 1 where the active ingredient is carporfen.

3. A product as in claim 2 where the carprofen is in the range of 0.1 to 10% by weight of composition.

4. A product as in claim 2 where the carporfen is in the range of 1 to 5% by weight of composition.

5. A product as in claim 2 wherein the said cosolvent is selected from one or more of the group consisting of polypropylene glycol, polyethylene glycol, glycol ether, glycerol, polyoxyethylene alcohol and esters of polyoxyethylene fatty acids and mixtures thereof.

6. A product as in claim 5 wherein the cosolvent is present in the range of 5 to 80% by weight of the composition.

7. A product as in claim 5 wherein the cosolvent is present in the range of 20% to 70% by weight of the composition.

8. A product as in claim 2 wherein the hydrophyllic gelling polymer is selected from the group consisting of CARBOPOL, acrylic polymers and copolymers, polyacrylamides or polysaccharides such as xanthan gum, acacia, tragacanth gum, guar gum, karaya gum, alginates and gelatin, modified celluloses such as carboxymethyl cellulose, sodiumhydroxypropyl methyl cellulose, hydroxyethyl cellulose and hydroxy propyl cellulose or mixtures thereof.

9. A product as in claim 8 wherein the hydrophyllic gelling polymer is present in the range of 0.1-20% by weight of the composition.

10. A product as in claim 8 wherein the hydrophyllic gelling polymer is present in the range of 0.2-10% by weight of the composition.

11. A product according to the claim 2 wherein there are added pharmaceutically acceptable excipients.

12. A product as in claim 11 wherein the excipients are selected from the group consisting of triethanol amine, sodium hydroxide, ammonium hydroxide, potassium hydroxide; flavoring agents, sweeteners, opacifying agents and buffering agents.

13. A method of treating pain and/or inflammation in an animal comprising oral administration of an oral semisolid composition using the formulations of claim 2.
